(19) United States
(12) Patent Application Publication
(10) Pub. No.: US 2025/0010110 A1
(43) Pub. Date: Jan. 9, 2025

(54) THE STAR HARNESS- A STABILIZER FOR CONTROLLING HIP FLEXION AND ABDUCTION

(71) Applicants: BOARD OF TRUSTEES OF THE UNIVERSITY OF ARKANSAS, Little Rock, AR (US); BIOVENTURES, LLC, Little Rock, AR (US); ARKANSAS CHILDREN'S NORTHWEST, INC., Springdale, AR (US)

(72) Inventors: Emma Herman, Edwardsville, IL (US); Hector Santillan, Russellville, AR (US); Jenova Kempkes, Fayetteville, AR (US); Adrienne Koder, Bentonville, AR (US); Samantha Blanchard, Fayetteville, AR (US)

(73) Assignees: BOARD OF TRUSTEES OF THE UNIVERSITY OF ARKANSAS, Little Rock, AR (US); BIOVENTURES, LLC, Little Rock, AR (US); ARKANSAS CHILDREN'S NORTHWEST, INC., Springdale, AR (US)

(21) Appl. No.: 18/763,942

(22) Filed: Jul. 3, 2024

Related U.S. Application Data
(60) Provisional application No. 63/524,814, filed on Jul. 3, 2023.

(51) Int. Cl. A62B 35/00

(52) U.S. Cl. CPC A62B 35/0018; A62B 35/0025

(57) ABSTRACT

Embodiments of the present disclosure pertain to a stabilizer that includes: (1) a first shoulder strap and a second shoulder strap that are each operational to be positioned on different shoulders of a subject; (2) a first hip strap and a second hip strap that are each operational to be positioned on different upper thighs of a subject and connectable to the first shoulder strap and the second shoulder strap, respectively; and (3) a connecting area operational to connect to the first shoulder strap, the second shoulder strap, the first hip strap and the second hip strap on the back of a subject. Additional embodiments of the present disclosure pertain to methods of applying the stabilizers of the present disclosure to a subject for various purposes, such as treating or preventing developmental dysplasia of the hip (DDH).

### CROSS-REFERENCE TO RELATED APPLICATIONS

This application claims priority to U.S. Provisional Patent Application No. 63/524,814, filed on Jul. 3, 2023. The entirety of the aforementioned application is incorporated herein by reference.

### BACKGROUND

A need exists for joint stabilizers that are more compact, less complex, and safer to use. Numerous embodiments of the present disclosure aim to address the aforementioned need.

### SUMMARY

In some embodiments, the present disclosure pertains to a stabilizer. The stabilizers of the present disclosure may include: (1) a first shoulder strap and a second shoulder strap that are each operational to be positioned on different shoulders of a subject; (2) a first hip strap and a second hip strap that are each operational to be positioned on different upper thighs of a subject and connectable to the first shoulder strap and the second shoulder strap, respectively; and (3) a connecting area operational to connect to the first shoulder strap, the second shoulder strap, the first hip strap and the second hip strap on the back of a subject.

In some embodiments, the stabilizers of the present disclosure may also include a first vertical strap connected to the first hip strap, and a second vertical strap connected to the second hip strap. In some embodiments, the first vertical strap and the second vertical strap are operational to pull the first hip strap and the second hip strap closer to the chest of the subject and thereby bring the hips of the subject closer to the chest of the subject.

In some embodiments, the stabilizers of the present disclosure may also include a first horizontal strap connected to the first hip strap, and a second horizontal strap connected to the second hip strap. In some embodiments, the first horizontal strap and the second horizontal strap are operational to pull the first hip strap and the second hip strap horizontally apart from each other and thereby widen the gap between the hips of the subject.

In some embodiments, the stabilizers of the present disclosure may also include a first leg strap and a second leg strap that are each operational to be positioned on different legs of a subject and connected to the first hip strap and the second hip strap, respectively.

Additional embodiments of the present disclosure pertain to a method of applying a stabilizer of the present disclosure to a subject. In some embodiments, the methods of the present disclosure may include: (1) placing the first shoulder strap and the second shoulder strap on different shoulders of a subject; (2) placing the first hip strap and the second hip strap on different upper thighs of the subject; (3) connecting the first hip strap and the second hip strap to the first shoulder strap and the second shoulder strap, respectively; and (4) connecting the first shoulder strap, the second shoulder strap, the first hip strap and the second hip strap to the connecting area on the back of the subject.

In some embodiments where the stabilizer includes a first vertical strap connected to the first hip strap and a second vertical strap connected to the second hip strap, the methods of the present disclosure also include a step of pulling the first vertical strap and the second vertical strap. In some embodiments, the pulling brings the first hip strap and the second hip strap closer to the chest of the subject and thereby brings the hips of the subject closer to the chest of the subject.

In some embodiments where the stabilizer includes a first horizontal strap connected to the first hip strap and a second horizontal strap connected to the second hip strap, the methods of the present disclosure also include pulling the first horizontal strap and the second horizontal strap. In some embodiments, the pulling pulls the first hip strap and the second hip strap horizontally apart from each other and thereby widens the gap between the hips of the subject.

In some embodiments where the stabilizer includes a first leg strap and a second leg strap, the methods of the present disclosure may also include a step of placing the first leg strap and the second leg strap on different legs of the subject. In some embodiments, the methods of the present disclosure also include a step of connecting the first leg strap and the second leg strap to the first hip strap and the second hip strap, respectively.

The methods of the present disclosure can have various applications. For instance, in some embodiments, the methods of the present disclosure may be used to treat or prevent a condition in a subject. In some embodiments, the condition to be treated or prevented includes, without limitation, developmental dysplasia of the hip (DDH), hip flexion, hip abduction, femur fractures, hip fractures, hip dislocations, osteoarthritis, congenital dysplasia, or combinations thereof.

### BRIEF DESCRIPTION OF THE DRAWINGS

A better understanding of the present invention can be obtained when the following detailed description is considered in conjunction with the following drawings, in which:

FIGS. 1A-1B provide illustrations of a stabilizer of the present on a subject.

FIGS. 2A-2B provide additional illustrations of a stabilizer of the present disclosure on a subject.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Numerous joint stabilizers exist for various applications. For instance, dysplasia of the hip (DDH) is a common neonatal condition. The American Academy of Orthopedic Surgeons estimates that, in the United States, 1 to 2 infants out of every 1,000 are born with DDH. Patients diagnosed with DDH are often treated between the ages of 1 day and 6 months old with a brace known as a Pavlik harness. This harness is used to help the dysplastic hip develop into a well-functioning joint. This brace is effective in most infants to prevent the need for invasive surgery. The brace is typically worn for 23 hours a day, for up to 12 weeks.

The Pavlik harness has a 63%-93% success rate, with compliance, severity, and age of diagnosis being notable factors contributing to the success rate. There are notable challenges related to this device, the first being that patients often experience skin breakdown from the harness, notably around the feet, neck, shoulders, or groin.

The second challenge related to the Pavlik harness involves the straps. The harness has straps that are adjusted by the orthopedic surgeon to properly position the angle of the infant's legs and hips. Parents are told not to adjust the straps or unbuckle these for any reason. Parents are also shown which buckles and straps they can adjust or unbuckle for routine infant care. However, parents or caregivers commonly unbuckle the wrong straps or adjust the ones they are not supposed to touch. Moreover, skin irritations are hard for parents and caretakers to understand, leading to the inadvertent maladjustment of straps or treatment noncompliance.

As such, a need exists for more effective stabilizers that are more compact, less complex, and safer to use. Numerous embodiments of the present disclosure aim to address the aforementioned need.

### Stabilizers

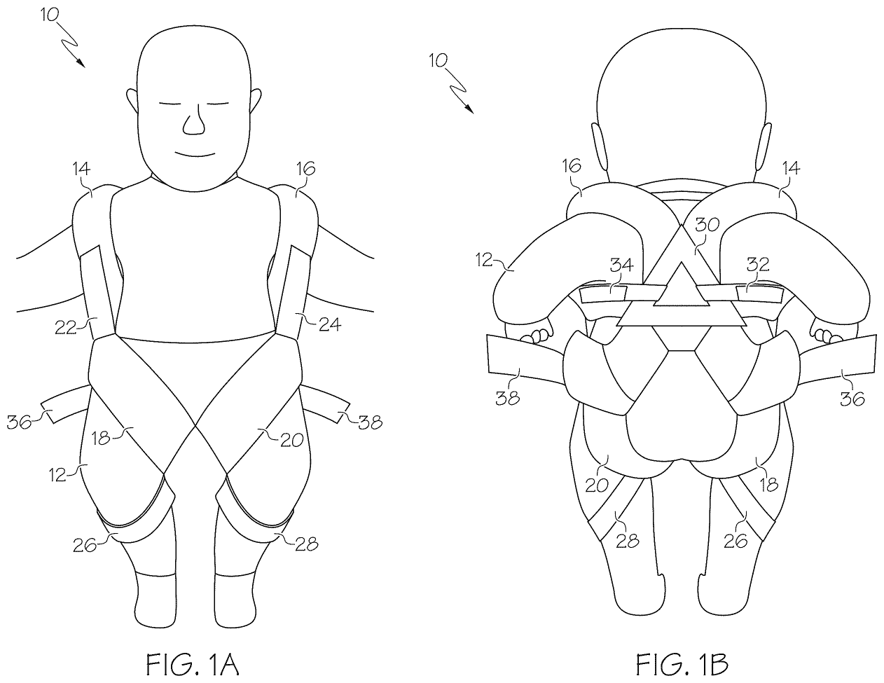

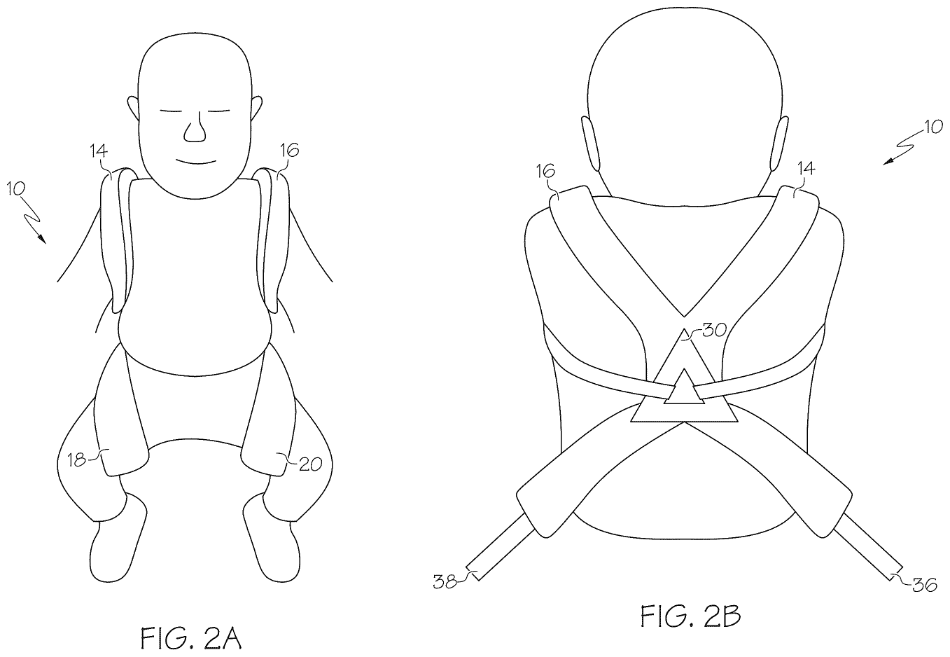

In some embodiments, the present disclosure pertains to a stabilizer. The stabilizers of the present disclosure may be illustrated herein as stabilizer 10 in FIGS. 1A-1B and 2A-2B. In some embodiments, stabilizer 10 includes a first shoulder strap 14 and a second shoulder strap 16. Each of the first shoulder strap 14 and the second shoulder strap 16 is operational to be positioned on different shoulders of a subject 12. In some embodiments, each of the first shoulder strap 14 and the second shoulder strap 16 is operational to be positioned on different shoulders of the subject while maintaining the chest area of the subject unobstructed.

In some embodiments, stabilizer 10 also includes a first hip strap 18 and a second hip strap 20. Each of the first hip strap 18 and the second hip strap 20 is operational to be positioned on different upper thighs of subject 12. Additionally, the first hip strap 18 and the second hip strap 20 are connectable to the first shoulder strap 14 and the second shoulder strap 16, respectively. In some embodiments the first hip strap 18 and the second hip strap 20 are connectable to the first shoulder strap 14 and the second shoulder strap 16 through one or more Velcro-based connections. In some embodiments, a Velcro-based connection is positioned between first shoulder strap 14 and first hip strap 18. In some embodiments, a Velcro-based connection is positioned between first shoulder strap 16 and first hip strap 20. In some embodiments, each of the first hip strap 18 and the second hip strap 20 is operational to be positioned on different upper thighs of the subject while maintaining the abdominal area unobstructed.

In some embodiments, stabilizer 10 also includes a connecting area 30. Connecting area 30 is operational to connect to the first shoulder strap 14, the second shoulder strap 16, the first hip strap 18 and the second hip strap 20 on the back of subject 12. In some embodiments, connecting area 30 is operational to connect to the first shoulder strap 14, the second shoulder strap 16, the first hip strap 18 and the second hip strap 20 on the back of subject 12 through one or more Velcro-based connections 32 and 34. In some embodiments, connecting area 30 is in the form of a triangle. In some embodiments, connecting area 30 also includes a protective cover. In some embodiments, the protective cover is operational to prevent tampering with the strap connections.

In some embodiments, stabilizer 10 also includes a first vertical strap 22 connected to first hip strap 18, and a second vertical strap 24 connected to a second hip strap 20. In some embodiments, first vertical strap 22 and second vertical strap 24 are operational to pull first hip strap 18 and second hip strap 20 closer to the chest of the subject and thereby bring the hips of the subject closer to the chest of the subject.

In some embodiments, stabilizer 10 also includes a first horizontal strap 36 connected to first hip strap 18, and a second horizontal strap 38 connected to second hip strap 20. In some embodiments, first horizontal strap 36 and second horizontal strap 38 are operational to pull first hip strap 18 and second hip strap 20 horizontally apart from each other and thereby widen the gap between the hips of the subject.

In some embodiments, stabilizer 10 also includes a first leg strap 26 and a second leg strap 28. In some embodiments, each of the first leg strap 26 and the second leg strap 28 is operational to be positioned on different legs of the subject and connected to the first hip strap 18 and the second hip strap 20, respectively.

The straps and connecting areas of the stabilizers of the present disclosure may include various compositions. For instance, in some embodiments, the compositions include a foam-based composition, such as an ethylene-vinyl acetate (EVA) foam. In some embodiment, the composition includes a polymer-based composition, such as a polyester mesh. In some embodiments, the composition includes a fabric-based composition, such as a bamboo fabric and/or a neoprene fabric. In some embodiments, the composition includes a Velcro-based composition.

### Methods of Applying a Stabilizer to a Subject

Additional embodiments of the present disclosure pertain to a method of applying a stabilizer of the present disclosure to a subject. With reference to stabilizer 10 in FIGS. 1A-1B and 2A-2B for illustrative purposes, the methods of the present disclosure may include: (1) placing the first shoulder strap 14 and the second shoulder strap 16 of stabilizer 10 on different shoulders of subject 12; (2) placing the first hip strap 18 and the second hip strap 20 on different upper thighs of the subject; (3) connecting the first hip strap 18 and the second hip strap 20 to the first shoulder strap 14 and the second shoulder strap 16, respectively; and (4) connecting the first shoulder strap 14, the second shoulder strap 16, the first hip strap 18 and the second hip strap 20 to the connecting area 30 on the back of the subject.

In some embodiments, connecting the first hip strap 18 and the second hip strap 20 to the first shoulder strap 14 and the second shoulder strap 16 occurs by adjusting one or more Velcro-based connections. In some embodiments, such a connection results in pulling of the legs into flexion at an angle of 90 degrees.

In some embodiments, connecting the first shoulder strap 14, the second shoulder strap 16, the first hip strap 18 and the second hip strap 20 to the connecting area 30 on the back of the subject occurs to achieve a desired angle of abduction. In some embodiments such a connection occurs by adjusting one or more Velcro-based connections 32 and 34.

In some embodiments, the methods of the present disclosure also includes a step of placing a protective cover on the connecting area 30. In some embodiments, the placement of the protective cover prevents tampering with the strap connections.

In some embodiments where the stabilizer includes a first vertical strap 22 connected to the first hip strap 18, and a second vertical strap 24 connected to the second hip strap 20, the methods of the present disclosure also include pulling the first vertical strap 22 and the second vertical strap 24. In some embodiments, the pulling brings the first hip strap and the second hip strap closer to the chest of the subject and thereby brings the hips of the subject closer to the chest of the subject.

In some embodiments where the stabilizer includes a first horizontal strap 36 connected to the first hip strap 18 and a second horizontal strap 38 connected to the second hip strap 20, the methods of the present disclosure include pulling the first horizontal strap 36 and the second horizontal strap 38. In some embodiments, the pulling pulls the first hip strap and the second hip strap horizontally apart from each other and thereby widens the gap between the hips of the subject.

In some embodiments where the stabilizer includes a first leg strap 26 and a second leg strap 28, the methods of the present disclosure also include a step of placing the first leg strap 26 and the second leg strap 28 on different legs of the subject. In some embodiments, the methods of the present disclosure also include a step of connecting the first leg strap 26 and the second leg strap 28 to the first hip strap 18 and the second hip strap 20, respectively.

The methods of the present disclosure can have various applications. For instance, in some embodiments, the methods of the present disclosure may be used to treat or prevent a condition in a subject. Additional embodiments of the present disclosure pertain to methods of treating or preventing a condition in a subject by placing a stabilizer of the present disclosure on the subject. In some embodiments, the stabilizer is placed on the subject in accordance with the placement methods of the present disclosure.

The methods of the present disclosure may be utilized to treat or prevent various conditions in a subject. For instance, in some embodiments, the condition to be treated or prevented includes, without limitation, developmental dysplasia of the hip (DDH), hip flexion, hip abduction, femur fractures, hip fractures, hip dislocations, osteoarthritis, congenital dysplasia, or combinations thereof. In some embodiments, the condition to be treated or prevented includes developmental dysplasia of the hip (DDH).

The stabilizers of the present disclosure may be applied to various subjects. For instance, in some embodiments, the subject is a human being. In some embodiments the subject is a pediatric subject. In some embodiments, the subject is an infant. In some embodiments, the subject is less than 12 months old. In some embodiments, the subject is around up to 9 months old.

### Advantages

The stabilizers of the present disclosure may provide various advantages. For instance, in some embodiments, the stabilizers of the present disclosure may be applied to a subject in a facile, expedited, convenient, and non-complex manner. Additionally, due to their compact nature, the stabilizers of the present disclosure may be applied to a subject without risk of skin irritation. For the same reasons, the stabilizers of the present disclosure are washable and storable in a more convenient manner.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Application of the Star Harness to Pediatric Patients

This Example illustrates the application of a stabilizer of the present disclosure (i.e., the Star Harness) to pediatric patients. As illustrated in FIGS. 1A-1B and 2A-2B, the Star Harness 10 is a medical device that can control hip flexion and abduction. The device consists of straps 14, 16, 18, and 20 that go around the patient's shoulders, back, hips, and upper thighs made of EVA foam that is encased in 100% polyester mesh. The mesh is covered with a charcoal grey colored bamboo fabric.

Since infants sleep on their back and are therefore at risk for a pressure injury, these straps 14, 16, 18 and 20 will connect in the back to a triangle 30 made of soft but durable neoprene fabric. The device will have additional straps 26 and 28 that go around the leg, below the knee, made of soft, non-stretch neoprene, that will prevent the infant from wiggling out of the device. The device will have Velcro straps that connect the shoulder straps 14 and 16 to the hip straps 18 and 20 in the front and back of the device. These will have a neoprene cover attached to them that can be snapped into place to cover the straps.

To apply the device, the infant will first be placed on top of the harness by the orthopedic surgeon, and their arms will be placed underneath the shoulder straps 14 and 16. Then, the legs will be placed into the hip straps 18 and 20 (e.g., left leg followed by the right leg). Next, the anterior (front) Velcro straps will be adjusted to pull the legs into flexion at an angle of 90 degrees. Thereafter, posterior Velcro straps 32 and 34 (the straps in the back of the device that connect to the hips) will be adjusted by the user for the desired angle of abduction. The user will then snap the neoprene cover over the strap to ensure that the caregiver or parent does not adjust the strap at home.

This design can allow users (e.g., physicians) to easily adjust the straps in a medical office. The design will also allow for parents to put on and take off the device without having to touch any of the Velcro straps. To take the infant out of the device at home, the parents can slip the arms out of the device and pull the device downwards and off. To place the infant back into the device, the caregiver can put the infant's legs in the device first and slide it up and over the shoulders in the back.

The device can come in four sizes: preemie, a small, a medium, and a large. The device can be placed on a child to ensure that they could not wiggle out of it with success. The device has applications in therapeutics, diagnostics, medical devices, digital health (software), and biological tools related to improving child health outcomes. For instance, beyond treating hip dysplasia, the Star Harness could also be used to treat infants with femur fractures and hip dislocations.

Moreover, the Star Harness is easy to use in comparison to competing products, not only for physicians, but for caretakers as well. The charcoal grey color of the fabric can hide stains well and leave materials looking fresh for longer. Furthermore, the bamboo fabric is eco-friendly and soft on the skin, preventing irritation and breakdown to vulnerable, high friction areas.

The device can also be machine washable, which is another benefit over competing solutions. Additionally, the device contains easily accessible and cheap materials, and is easy to replicate for manufacturing purposes. Finally, the device utilizes existing CPT codes (e.g., follows HCPCS code L1620, CPT 97760, and 97762).

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A stabilizer comprising:
a first shoulder strap and a second shoulder strap, wherein each of the first shoulder strap and the second shoulder strap is operational to be positioned on different shoulders of a subject;
a first hip strap and a second hip strap, wherein each of the first hip strap and the second hip strap is operational to be positioned on different upper thighs of the subject, and wherein the first hip strap and the second hip strap are connectable to the first shoulder strap and the second shoulder strap, respectively; and
a connecting area, wherein the connecting area is operational to connect to the first shoulder strap, the second shoulder strap, the first hip strap and the second hip strap on the back of the subject.

2. The stabilizer of claim 1, wherein each of the first shoulder strap and the second shoulder strap is operational to be positioned on different shoulders of the subject while maintaining the chest area of the subject unobstructed.

3. The stabilizer of claim 1, wherein each of the first hip strap and the second hip strap is operational to be positioned on different upper thighs of the subject while maintaining the abdominal area unobstructed.

4. The stabilizer of claim 1, wherein the first hip strap and the second hip strap are connectable to the first shoulder strap and the second shoulder strap through one or more Velcro-based connections.

5. The stabilizer of claim 1, further comprising a first vertical strap connected to the first hip strap, and a second vertical strap connected to the second hip strap, wherein the first vertical strap and the second vertical strap are operational to pull the first hip strap and the second hip strap closer to the chest of the subject and thereby bring the hips of the subject closer to the chest of the subject.

6. The stabilizer of claim 1, further comprising a first horizontal strap connected to the first hip strap, and a second horizontal strap connected to the second hip strap, wherein the first horizontal strap and the second horizontal strap are operational to pull the first hip strap and the second hip strap horizontally apart from each other and thereby widen the gap between the hips of the subject.

7. The stabilizer of claim 1, further comprising a first leg strap and a second leg strap, wherein each of the first leg strap and the second leg strap is operational to be positioned on different legs of the subject and connected to the first hip strap and the second hip strap, respectively.

8. The stabilizer of claim 1, wherein the connecting area is operational to connect to the first shoulder strap, the second shoulder strap, the first hip strap and the second hip strap on the back of the subject through one or more Velcro-based connections.

9. The stabilizer of claim 1, wherein the connecting area is in the form of a triangle.

10. The stabilizer of claim 1, wherein the connecting area further comprises a protective cover, wherein the protective cover is operational to prevent tampering with the strap connections.

11. A method of applying a stabilizer to a subject, wherein the stabilizer comprises:
a first shoulder strap,
a second shoulder strap,
a first hip strap,
a second hip strap, and
a connecting area; and wherein the method comprises:
placing the first shoulder strap and the second shoulder strap of the stabilizer on different shoulders of the subject;
placing the first hip strap and the second hip strap on different upper thighs of the subject;
connecting the first hip strap and the second hip strap to the first shoulder strap and the second shoulder strap, respectively; and
connecting the first shoulder strap, the second shoulder strap, the first hip strap and the second hip strap to the connecting area on the back of the subject.

12. The method of claim 11, wherein connecting the first hip strap and the second hip strap to the first shoulder strap and the second shoulder strap results in pulling of the legs into flexion at an angle of 90 degrees.

13. The method of claim 11, wherein connecting the first hip strap and the second hip strap to the first shoulder strap and the second shoulder strap occurs by adjusting one or more Velcro-based connections.

14. The method of claim 11, wherein connecting the first shoulder strap, the second shoulder strap, the first hip strap, and the second hip strap to the connecting area on the back of the subject occurs to achieve a desired angle of abduction.

15. The method of claim 11, wherein connecting the first shoulder strap, the second shoulder strap, the first hip strap, and the second hip strap to the connecting area on the back of the subject occurs by adjusting one or more Velcro-based connections.

16. The method of claim 11, further comprising a step of placing a protective cover on the connecting area to prevent tampering with the strap connections.

17. The method of claim 11, wherein the stabilizer further comprises a first vertical strap connected to the first hip strap, and a second vertical strap connected to the second hip strap, and wherein the method further comprises:
pulling the first vertical strap and the second vertical strap,
wherein the pulling brings the first hip strap and the second hip strap closer to the chest of the subject and thereby brings the hips of the subject closer to the chest of the subject.

18. The method of claim 11, wherein the stabilizer further comprises a first horizontal strap connected to the first hip strap, and a second horizontal strap connected to the second hip strap, and wherein the method further comprises:
pulling the first horizontal strap and the second horizontal strap,
wherein the pulling pulls the first hip strap and the second hip strap horizontally apart from each other and thereby widens the gap between the hips of the subject.

19. The method of claim 11, wherein the stabilizer further comprises a first leg strap and a second leg strap, and wherein the method further comprises:
placing the first leg strap and the second leg strap on different legs of the subject; and
connecting the first leg strap and the second leg strap to the first hip strap and the second hip strap, respectively.

20. The method of claim 11, wherein the method is used to treat or prevent a condition in a subject.

21. The method of claim 20, wherein the condition is selected from the group consisting of developmental dysplasia of the hip (DDH), hip flexion, hip abduction, femur fractures, hip fractures, hip dislocations, osteoarthritis, congenital dysplasia, or combinations thereof.

22. The method of claim 21, wherein the condition is developmental dysplasia of the hip (DDH).

23. The method of claim 21, wherein the subject is a pediatric subject.

24. The method of claim 21, wherein the subject is less than 12 months old.
